(19) United States
(12) Patent Application Publication
(10) Pub. No.: US 2010/0081839 A1
(43) Pub. Date: Apr. 1, 2010

(54) PROCESS FOR PREPARATION OF CRYSTALLINE CLOPIDOGREL HYDROGEN SULPHATE FORM I

(75) Inventors: Ashok KUMAR, Mumbai (IN); Priti Jayesh Bhayani, Maharashtra (IN); Vaibhav Chinubhai Doshi, Mumbai (IN); Ashvini Saxena, Ratllam (IN); Gunjan Pramod Pathak, Ratlam (IN); Rashmi Abhyankar, Ratlam (IN); Sarvanan Manavalan, Ratlam (IN); Mukesh Purohit, Ratlam (IN); Santosh Parihar, Ratlam (IN)

Correspondence Address: BLANK ROME LLP, WATERGATE, 600 NEW HAMPSHIRE AVENUE, N.W., WASHINGTON, DC, 20037, US

(73) Assignee: IPCA Laboratories Ltd., Mumbai (IN)

(21) Appl. No.: 12/509,145

(22) Filed: Jul. 24, 2009

(30) Foreign Application Priority Data
Jan. 29, 2007 (IN) 161/MUM/2007
Feb. 13, 2007 (IN) 292/MUM/2007
Aug. 20, 2007 (IN) 1594/MUM/2007
Jan. 7, 2008 (IN) 43/MUM/2008
Jan. 29, 2008 (IN) PCT/IN2008/000052

(51) Int. Cl. C07C 309/19

(52) U.S. Cl. 562/100

(57) ABSTRACT

The present invention describes an improved industrial process for crystallizing out polymorph ‘Form I’ of (+) clopidogrel hydrogen sulphate (also called clopidogrel bisulphate) in) from either a Type-1 solvent or liquid characterized by comprising two or more functional groups or their mixtures thereof; or a Type-II solvent and/or solvent mixture selected from the group of methyl ethyl ketone, cyclopentylmethyl ether, dipropylglycolether, dibutylglycol ether, propylmethyl cellosolve, butylmethylcellosolve, propylethylellosolve, butylethylcellosolve or their cross combinations in a reproducible manner without detectable contamination of polymorph designated as ‘Form II’. The invention also discloses improvements in the preparation of clopidogrel free base and process for recovery and recycling of resolving agent camphor sulphonic acid.

### RELATED REFERENCE

This patent application claims priority from our Indian patent applications: 161/MUM2007 filed on Jan. 29, 2007; 292/MUM/2007, filed on Feb. 13, 2007; 1594/MUM/2007, filed on Aug. 20, 2007; and 43/MUM/2008, filed on Jul. 1, 2008. The contents of which may be treated as incorporated herein by reference.

### TECHNICAL FIELD OF INVENTION

The present invention relates to a process for preparing (+)-(S)-alpha-2-(chlorophenyl)-6,7-dihydrothieno[3,2-C]pyridine-5-(4-H)-acetic acid methyl ester hydrogen sulphate of Formula I, commonly known as Clopidogrel bisulphate in “Form-I” crystalline form. The present invention further relates to improvements in the preparation of clopidogrel free base and process for recovery and recycling of resolving agent camphor sulphonic acid.

### BACKGROUND OF THE INVENTION

(+)-(S)-alpha-2-(chlorphenyl)-6,7-dihydrothieno-[3,2-C]-pyridine-5-(4-H)-acetic acid methyl ester known as clopidogrel under the international Non-Proprietary Name is marketed as hydrogen sulphate salt. Clopidogrel is known for its platelet aggregating and anti-thrombotic properties and finds medicinal applications in this field. It can be represented by Formula-I, and was disclosed in U.S. Pat. No. 4,529,596 (hereinafter referred as ‘596’ patent) in its racemic form for the first time.

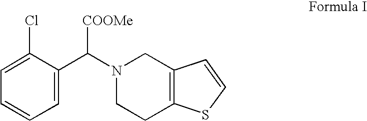

The pure enantiomeric forms of clopidogrel (dextro and levo isomers) were disclosed in EP 281459 which teaches the isolation of the dextro rotatory isomer of Clopidogrel by diasteriomeric salt formation of racemic Clopidogrel base using an optically active acid such as L-camphor-10-sulfonic acid.

The pure dextrorotatory isomer of clopidogrel (herein after referred as clopidogrel free base) was released from the respective diastereomeric salt by reaction with sodium bicarbonate in an aqueous solvent followed by extractive work-up. The starting racemic clopidogrel free base was obtained from hydrogen sulphate salt of racemic clopidogrel by following an aqueous treatment in presence of bicarbonate.

The Clopidogrel free base was then converted into its hydrogen sulfate salt by dissolving in acetone, cooling and mixing with concentrated sulfuric acid to precipitation. The precipitate thus obtained is then isolated by filtration, washed and dried to give Clopidogrel hydrogen sulfate in the form of white crystals whose melting point was 184° C. and optical rotation was +55.1° (c=1.891/CH₃OH). But '459 patent did not characterize or suggest any name to this crystals (polymorph) of Clopidogrel hydrogen sulfate.

Subsequently International patent publication, WO 99/65915 (herein after referred as '915 patent), disclosed two polymorphic forms of Clopidogrel hydrogen sulfate referred to as Form-I and Form-II. The '915 patent identified that the precipitation method described in '459 patent had led to crystalline Form-I. The '915 also deals with a new crystalline form palled Form-II of Clopidogrel hydrogen sulfate. The latter is suggested to be thermodynamically most stable crystalline form. According to '915 patent both polymorphs, namely Form I and Form II, were prepared from the same solvent viz; acetone. In this publication, the process to obtain clopidogrel free base from camphor sulphonate salt was carried out in aqueous reaction conditions in presence of potassium carbonate followed by extractive work-up.

The process for obtaining crystalline Form-I of Clopidogrel hydrogen sulfate according to example 1A of the '915 patent describes the introduction of Clopidogrel camphor sulfonate in methylene dichloride (MDC) and transformation of salt into the clopidogrel base with potassium carbonate in aqueous conditions. Clopidogrel base is extracted in MDC and solvent is evaporated. Residue obtained is dissolved in acetone and cooled.

Addition of sulfuric acid to said solution precipitate out Clopidogrel hydrogen sulfate in Form-I. Also in the same application it was described to get Form-II either by keeping mother liquor of Form-I for prolonged periods or by heating the acetone solution containing the base after addition of sulfuric acid to reflux or by subjecting the suspension to mechanical shearing using a shearing device or by inoculation.

However, this process was not found to be suitable for the production of Form I of clopidogrel hydrogen sulphate on an industrial scale owing to its thermodynamic instability in solvents like acetone and invariably yielded Form II without having the need of keeping for longer periods (ref. '915 patent). This problem became the subject of many latter patent applications and a detailed study of the various publications clearly indicates that manufacture of Form I of clopidogrel hydrogen sulphate poses a well known technical challenge to the process chemist. Although most of the general class of organic solvents are said to be used for preparation of Form I polymorph, it appears both from the latest literature and from the experimental studies on this polymorph by the present authors, most of these solvents gives only Form II or a Form I contaminated with Form II. The presence of Form II in form I can lead to instability of Form I which results in inconsistency in formulations and ultimately leading to varying drug bioavailability. Moreover, among all these, most processes are not reliably reproducible on large scale.

The present inventors have also noted that, since the Form I is thermodynamically unstable, the process variants of dissolving clopidogrel hydrogen sulphate salt in conventional solvents at higher temperature and cooling to precipitate Form I resulted in Form II or its mixture with Form I. Moreover, the poor solubility of clopidogrel salt (whereas the free base possess good solubility) in most of the known solvents does not allow to use this crystallization process variant to be practiced.

There are ample literatures available for preparation of Form I, such as US2006074242, US20060205766, WO2005117866, WO2005100364, US2005059696, WO2005063708, WO2005016931, WO2005003139, SK12852002, P1355514, WO2004026879, WO2004081015, WO2004020443, WO2004081016, WO2004048385, WO2004081016, WO20040024012, WO2006087226, US20030114479, US2003225129, & WO2002059128 a few to name, but it is also clear from these literature that same solvent unarguably gives two different crystalline forms in different hands, which is surprising, for example, the US2003225129 patent (herein after referred as '129 patent) describes process for the preparation of Form-H from solvents selected from dichloromethane, 1,4-dioxane, toluene, chloroform, ethyl acetate, methyl ethyl ketone and t-butyl methyl ether. The '129 patent, for the first time, claimed to produce Form II from ethyl acetate which was the main subject of WO2004020443 patent application for Form I preparation. A cursory review of the prior art indicates that almost all classes of solvents covering ethers, diethers, esters, alcohols, nitriles, ketones etc. are disclosed for use in the preparation of Form I, but those solvents are disclosed also taught to give Form II in the same or different publication.

WO2005003139 patent discusses combination of polar and non-polar solvent combinations for obtaining Form I, however, the present inventors have found that the adjustment of specific proportion of two or more solvents are rather difficult and does not give consistent & reproducible results while adjusting the polarity of mixtures.

So, it is evident from the prior art that the methods to produce Form-I of clopidogrel hydrogen sulphate from known solvents are poorly reproducible, necessitating the optimization of experimental conditions and/or the selection of suitable solvents. Since Form-I is kinetically controlled and Form-II is thermodynamically controlled form, they require very specific temperature range and specific conditions for getting reproducible results in conventional solvents. Also, in these solvents a minor variation in condition appears to give Form-II instead of expected Form-I or a mixture of Form-I & Form-II. Since, Form I of clopidogrel hydrogen sulphate is used for pharmaceutical formulation, the importance of a rugged method that gives Form I consistently doesn't require any emphasis.

As for obtain the clopidogrel free base from clopidogrel acid salts, there are few literatures disclose alternative bases other than alkali metal carbonates reported in either EP281459 or WO99/65915. For example, WO2004074215 disclosed a racemization process for R-clopidogrel acid salt using liquor ammonia and finally aqueous extractive work-up to isolate the clopidogrel free base. A parallel publication, WO/2004/108665, on process for clopidogrel has used an aqueous extractive work-up to isolate clopidogrel free base from it's acid salt. Another patent application No. WO/2007/032023 also discloses ammonia for basification of clopidogrel salt using an aqueous extractive work-up to produce clopidogrel free base.

In parallel to the above reports on the preparation of clopidogrel, the applicants own publication, WO/2005/104663, disclosed the use of ammonia in the preparation of clopidogrel free base from clopidogrel salt, especially camphorsulphonate salt using an aqueous extractive work-up.

According to our search, in all the preparative methods disclosed for clopidogrel, the use of either alkali metal carbonates or bicarbonates for the breaking of clopidogrel acid addition salt with an aqueous extractive work-up is common. Although ammonia is used for salt breaking, however an aqueous condition/work-up was used. Clopidogrel being an oily substance, it is advantageous to convert to an acid addition salt for better handling and storage and therefore, its conversion to a salt form is almost unavoidable for stable storage. Clopidogrel has a methyl ester group as evidenced from the structure of Formula I. The clopidogrel when hydrolyzed it forms an impurity named Clopidogrel free acid of Formula X, which needs to be monitored in the active substance as per pharmacopoeial specification. The main cause of generation of this impurity in clopidogrel is the hydrolysis of methyl ester under aqueous conditions during the processing.

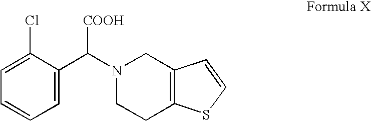

In order to control the hydrolysis of ester, very mild bases like bicarbonates and controlled reactions conditions were employed in the art. Due to the poor solubility of either clopidogrel acid addition salt or clopidogrel free base in water, the process of breaking the salt in aqueous conditions were not effective/fast leading to larger reaction time which is detrimental to product purity. Thus, carrying out reactions at higher temperatures or for longer period lead to formation of acid impurity which necessitates extra purification resulting into yield losses and increase in number of operations that are not desirable for a practical process.

Owing to the economy of the process, the expensive resolving agent ‘camphor sulphonic acid’ should be recovered and recycled after resolution, which is almost impractical in reported processes because of its high solubility in aqueous solutions. There are many publications disclosing different processes for recovering camphor sulphonic acid after its use as resolving agent, for example EP149885, JP08325262, CN1265391, and CN1267664; however, in aqueous conditions. Being highly soluble in water, the recovery from aqueous reaction solution is very difficult and in most cases requires column purification, which is not economically viable. Thus, the search for a manufacturing process for the preparation of Clopidogrel resulting in a satisfactory yield/purity of final product, especially Form I polymorph, remains undoubtedly of interest.

### OBJECTIVES OF THE PRESENT INVENTION

An objective of the present invention is to provide solvent systems where the Form I crystals of clopidogrel hydrogen sulphate can be efficiently and reproducibly formed and easily maneuvered at large scale operations.

It is also an objective of the present invention to discover an efficient process to recover and recycle camphor sulphonic acid, apart from simplifying the process for clopidogrel.

### SUMMARY OF THE INVENTION

Thus in one aspect, the present invention provides solvent(s) or solvent systems useful for making Form I clopidogrel hydrogen sulphate (Formula IB) in a consistent & reproducible manner and processes for the same.

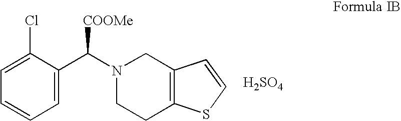

Accordingly, the present invention provides an improved industrial process for crystallizing out polymorph ‘Form I’ of (+) clopidogrel hydrogen sulphate (also called clopidogrel hydrogen sulphate or clopidogrel bisulphate) from a Type-I organic solvent or liquid comprising two or more functional groups with a proviso that at least one functional group is different from each other; or from a Type-II solvent and/or solvent mixture selected from methyl ethyl ketone, cyclopentylmethyl ether, dipropylglycolether, dibutylglycol ether, propylmethyl cellosolve, butylmethylcellosolve, propylethylellosolve, butylethylcellosolve or their mixtures, in a reproducible manner without detectable contamination of form II. Examples of multiple functional groups which can be present in the Type-I solvent are selected from those of ketone-ester, ether-ketone, nitrile-ester, alcohol-ester, alcohol-ether, ketone-ether, halogen substituted esters, nitro-esters, ketone-nitrile etc. Especially preferred Type-I solvents possess carbon atoms ranging from C4 to C12 atoms, specific examples of such solvents are ethyl acetoacetate, methyl acetoacetate, chloropropionyl acetate, alkyl lactates, chloroethylacetoacetate, chloroacetylacetoacetate etc.

In a preferred embodiment of the present invention, the process for preparation of ‘Form I’ comprises dissolving the clopidogrel base in either Type-I or Type-II solvent as discussed/defined above; cooling the obtained clopidogrel base solution to a temperature of −20 to 30° C.; adding concentrated sulphuric acid (98%, d=1.84) while maintaining the temperature in the above range to obtain clopidogrel hydrogen sulphate salt; optionally further maintaining the salt at a temperature of about 10 to 30° C. and filtering the crystals of Form I obtained.

In a second embodiment of the present invention, the process for preparation of ‘Form I’ comprises dissolving the clopidogrel hydrogen sulphate of any crystal form, for example, amorphous, Form II, or Form III, or their mixtures or contaminated Form I of clopidogrel hydrogen sulphate in a suitable multifunctional group solvent (Type-I) as defined above; then cooling the obtained clopidogrel hydrogen sulphate solution to a temperature of −20 to 30° C. and maintaining the salt at a temperature of about 10 to 30° C. to complete precipitation of Form I and filtering the crystals of Form I obtained.

In a second aspect, the present invention provides an improved process for preparing clopidogrel free base from its acid addition salt. The present process comprises the step of treating clopidogrel acid salt in non-aqueous conditions using ammonia to obtain clopidogrel in free base form.

In one embodiment of the invention the process according to the invention comprises treating clopidogrel acid salt, for example, bisulphate, hydrochloride, hydrobromide, camphorsulphonate salt or similar salts with ammonia in a non-aqueous solvent. The clopidogrel free base can be recovered by filtering out the ammonium salt of acid from the organic solvent, followed by elimination of solvent.

In a third aspect, the present invention provides an efficient process to recover and recycle camphor sulphonic acid after its use in the resolution of a racemic substrate, for example, (±)-clopidogrel, comprises:

a) treating the diastereomeric camphor sulphonate salt of substrate with ammonia in a non-aqueous reaction solvent;
b) separating the ammonium camphor sulphonate salt from the substrate;
c) treating said ammonium camphor sulphonate salt with an acid in non-aqueous solvent to exchange the ammonium ion with the acid to free camphor sulphonic acid; and
d) isolating free camphor sulphonic acid from the reaction for recycling.

The details of one or more embodiments in the practice of the inventions are set forth in the description below. Other features, objects and advantages of the inventions will be apparent from the appended examples and claims.

### BRIEF DESCRIPTION OF FIGURES

FIG. 1 represents Powder X-Ray diffraction pattern (PXRD) of clopidogrel hydrogen sulphate Form I prepared according to example 1 of the present invention.

FIG. 2 represents Differential Scanning Calorimetry record of Form I of clopidogrel hydrogen sulphate prepared according to example 1 of the present invention.

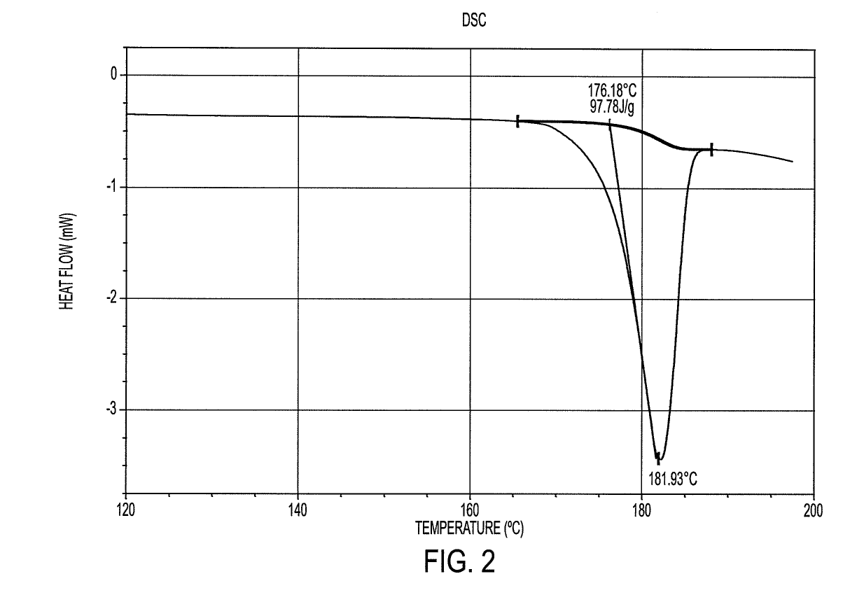

FIG. 3 represents Powder X-Ray diffraction pattern (PXRD) of clopidogrel hydrogen sulphate Form I standard as given in '915 patent.

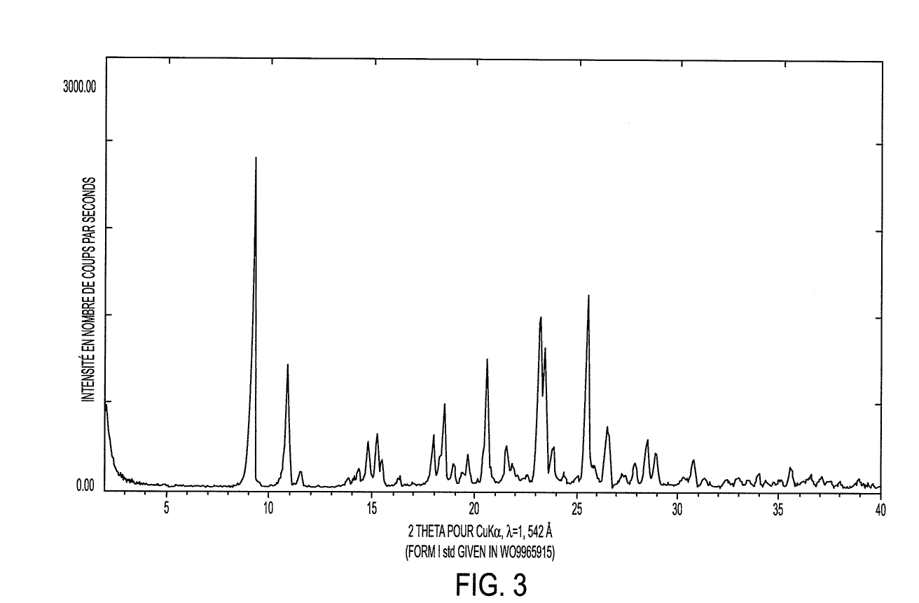

FIG. 4 represents the spectrogram obtained by Fourier Transform Infra Red spectrometry (FTIR) of clopidogrel hydrogen sulphate Form I prepared according to example 1 of the present invention.

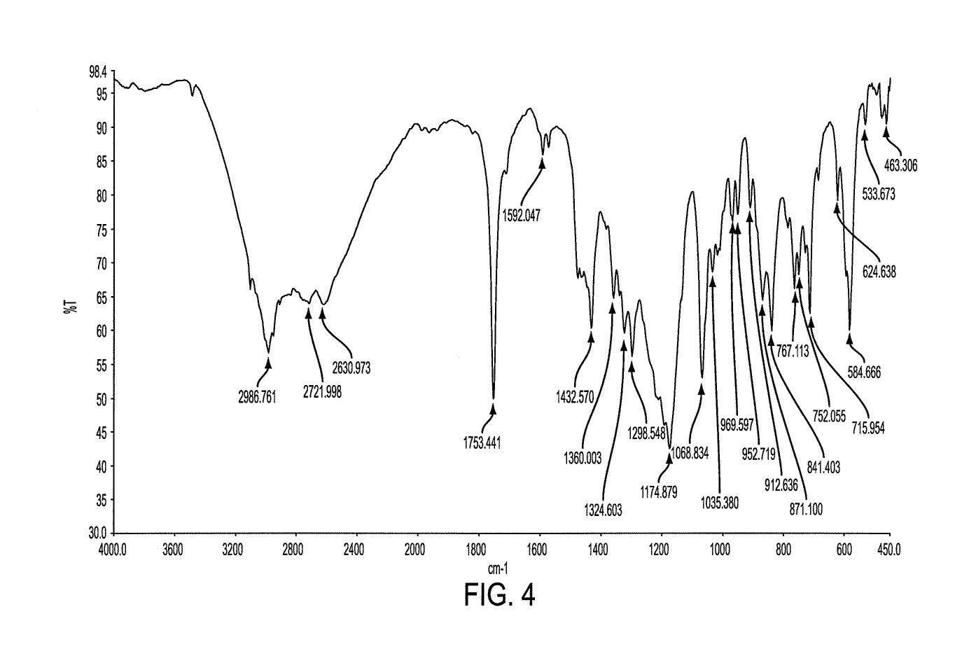

FIG. 5 represents an overlay of powder x-ray diffraction pattern (PXRD) of clopidogrel hydrogen sulphate crystalline Form I and Form II.

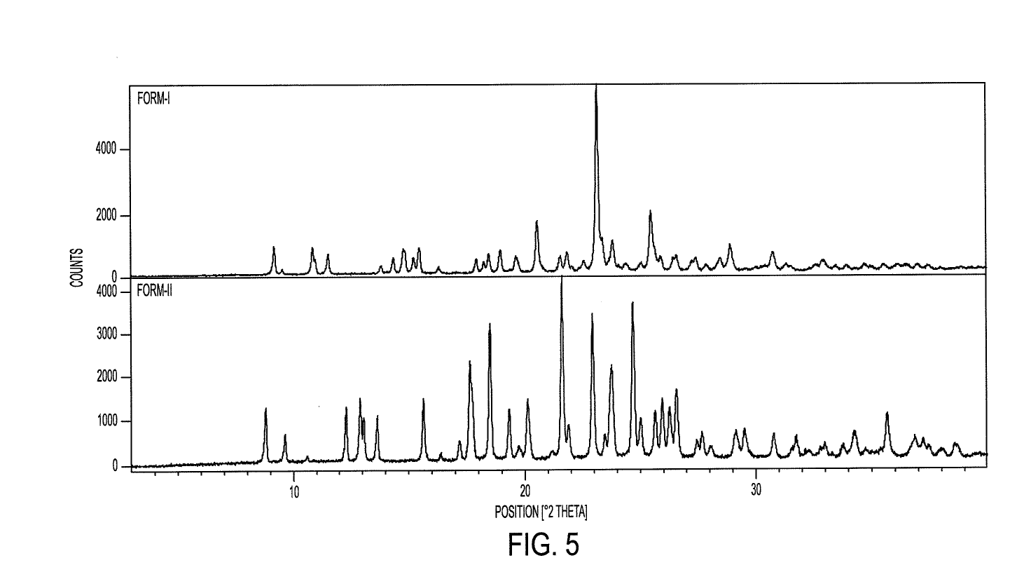

### DETAILED DESCRIPTION OF THE INVENTION

Unless specified otherwise, all technical and scientific terms used herein have the same meaning as commonly understood by one of ordinary skill in the art, to which this invention belongs. Although any methods and materials similar or equivalent to those described herein can be used in the practice or testing of the present invention the preferred methods and materials are described. To describe the invention, certain terms are defined herein specifically as follows.

Unless stated to the contrary, any of the words “including,” “includes,” “comprising,” and “comprises” mean “including, without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

The term “isolating” is used to indicate separation or collection or recovery of the compound being isolated in the specified crystalline form.

The term “separating from a solvent” with respect to the crystalline solids described herein means obtaining a solid of specified characteristics from a solution or a partial solution.

The term “treating” means adding or combining or mixing the stated reagent or materials to the things being treated.

As used herein the term “(+)-clopidogrel” or “(+)-(S)-clopidogrel” means the dextro-rotatory isomer of clopidogrel. The name “Clopidogrel hydrogen sulphate” or “clopidogrel bisulphate” used means the compound represented in formula IB.

The term “forming a solution” means obtaining a solution of a substance in a solvent in any manner.

The term “non-aqueous medium” means a solvent medium that does not contain water in significant amounts. The term does not exclude solvents containing insignificant amounts of water, which may be less than 5%, more preferably less than 2%.

The term “inoculating” has the same meaning as the term “seeding,” and means adding previously obtained solid to facilitate crystallization. Thus, the term “seeding crystals” with respect to claimed process means inoculating crystals/powder of previously obtained crystalline Form I of clopidogrel hydrogen sulphate.

It should be understood that there exists equilibrium between a free species and salt form of a compound capable of forming salt with bases/acids (e.g., by virtue of having an amino-functionality in the molecule).

For the purposes of this description and claims of the present invention, the term “crystalline Form I” or “Form I” of clopidogrel hydrogen sulphate is the polymorphic form denoted as Form I and having characteristics as given in U.S. Pat. No. 6,429,210. Identification of solids obtained by the process of the invention can be made by comparing with the reference analytical information provided in the U.S. Pat. No. 6,429,210. Of course, it should be understood that operator, instrument and other similar variables may result in some margin of error with respect to analytical characterization of the solid.

Large scale production of Form I according to the process described in the '915 patent or '210 patent resulted in Form II or a mixture with Form I or contaminated with other forms at a higher percentage. This led us to find suitable solvents or conditions where these problems are minimized and ensure reproducibility of Form I, without contamination of other forms, especially Form II. This research has led to an efficient process where Form I can be produced reliably from a single solvent or mixture of solvents.

Thus the present inventors, on exploring various process alternatives, for a reliable process solution have found that the use of selected organic solvents which are categorized into either Type-I or Type-II solvents, wherein the Type-I organic solvent possesses two or more functional groups, wherein at least one such functional group is distinctly different from other or the cross mixtures of such solvents; and the Type-II solvent is a solvent selected from methyl ethyl ketone, cyclopentylmethyl ether, dipropylglycolether, dibutylglycol ether, propylmethyl cellosolve, butylmethylcellosolve, propylethylellosolve, butylethylcellosolve or their mixtures, for crystallization of clopidogrel permits reliable preparation of Form I of clopidogrel hydrogen sulphate. Especially preferred functional groups in the Type-I solvent are either ester-ketone, or ether-ketone, or hydroxyl-esters, keto-esters, or ester-nitrile, or ether-ester, or nitro-esters etc. Especially preferred Type-I solvent has carbon atoms from C4 to C12 in the molecule. The preferred Type-I solvents are ethyl acetoacetate, methyl acetatoacetate, alkyl lactate, chloropropylacetate esters. 4-chloroethylacetoacetate esters, chloroacetyl acetoacetate esters etc.

It has also been observed that presence of a specified concentration range of other specific solvent(s) in the novel solvent(s) of the present invention does not alter results of the present process. Thus a solvent selected from the group of esters, C3-C6 aliphatic or alicylcic ketones, C3-C6 chain or branched chain alcohols, and ethers in about 1 to 25 weight percent in the Type-I (possessing multi-functional group) or Type-II solvents of the present invention gives Form I consistently. The weight percent will depend upon the choice of the individual solvent from the above class of solvents, but can be determined by routine experimentation. Especially preferred solvents for making mixtures are acetone, methylpropyl ketone, methylisopropyl ketone, isobutyl ketone among ketones; propylacetate and butyl acetate among esters; butanol and pentanol among alcohols; and tertiary butyl methyl ether and cyclopentyl methyl ether among ethers. Most of these other solvents are not suitable independently for preparing pure Form I. Sometimes, the presence of these solvents in the process of the present invention are especially useful in removing the solvents of multiple functional groups used in the invention to keep their level in the final product as low as possible to meet the pharmacopoeias specifications. It is very surprising to note that the reactive compounds like ethyl acetoacetate is inert through out the crystallization process of the present invention and does not participate in any significant chemical change or impurity formation as against the expected reactivity of such solvents.

Accordingly, in one embodiment, the present invention provides a process for preparing polymorph Form I of clopidogrel hydrogen sulphate comprising dissolving the (+)-clopidogrel base in either a Type-I or Type-II organic solvent or their mixtures, as defined/described before; cooling the obtained solution to a temperature of, preferably, −25° C. to 30° C.; adding concentrated sulphuric acid (98%, d=1.84) by maintaining the temperature preferably in the range of −20 to 30° C.; and maintaining for a period of 5 to 10 hours to crystallize out the Form I clopidogrel hydrogen sulphate. The precipitated crystals may be collected by conventional methods like filtration or centrifugation. Although temperature lower than −20° C. also works while addition of sulfuric acid, owing to the industrial applicability the above range is preferred.

A mixture of Type-I or Type-II solvents can be mixed with each other and may be employed in all proportions.

In the process for preparation of Form I, the preferred concentration of sulphuric acid is in the range of 80% to 98% and the molar ratios are in the range of 1 to 1.1 with respect to the (+)-clopidogrel base. The most preferred concentration of sulphuric acid used in the salt formation is 90-98%. The sulphuric acid may be employed directly in the salt formation or may be employed as a solution in a carrier solvent. Especially preferred carrier solvents, although not limited to, are those selected from the above described Type-I or Type-II group solvents. In a preferred embodiment of the process, the exotherm of sulphuric acid addition is controlled by cooling and maintaining the temperature in between −10 to 10° C.

The Form I so obtained was characterized by PXRD, DSC and FTIR without any detectable quantity of Form II or other polymorphic Forms with respect to the standard PXRD pattern of Form I as described in '915 patent. The Form-I obtained by the process of the present invention does not contain any detectable Form-II polymorph and therefore it is stable to storage/handling.

In the process, the clopidogrel free base solution may be obtained by dissolving clopidogrel free base in the solvent at any temperature at or below the reflux temperature of the solvent and the solution may then be filtered to remove any particulate matter. Once the solution of the clopidogrel free base is obtained, the solution is cooled to a temperature below 25 degree, more particularly below 10 degrees and sulphuric acid solution is incorporated to form the clopidogrel hydrogen sulphate salt. The mass is then cooled or maintained at this temperature until crystallization of the solid is complete. The solid is filtered, washed, and dried. In the process, optionally, either before or after sulphuric acid addition, the solution may be seeded (inoculated with seed crystals) with previously obtained crystals of the Form I. The seed crystals may be obtained by performing the present invention at a lower scale or by methods known in the art. The process conditions are further illustrated in the Examples.

In a second embodiment of the present invention, a process for preparation of ‘Form I’ is provided which comprises dissolving the clopidogrel hydrogen sulphate salt of any crystal form (for example, amorphous, Form II, or Form III, or their mixtures or contaminated Form I in a Type-I organic solvent containing suitable multi-functional group as discussed above, for example, ethyl acetoacetate or alkyl lactate, or chloroethylacetoacetate, then cooling the obtained clopidogrel hydrogen sulphate solution to a temperature of −20 to 30° C. and maintaining the salt at a temperature of about 10 to 30° C. to complete precipitation of Form I and filtering the crystals of Form I obtained. The dissolution of the clopidogrel salt in the solvent is carried out at a temperature from ambient to reflux temperature of the solvent, preferably from 50 to reflux temperature of the solvent. The solution is then allowed to cool to room temperature, optionally kept for a holding time and then cooled to 0 to 5 degree to precipitate the Form I clopidogrel hydrogen sulphate.

The starting material, clopidogrel free base may be obtained by following any known process disclosed in the literature.

The present inventors provide an efficient process to obtain clopidogrel free base from an acid addition salt, especially camphor sulphonate salt of clopidogrel, wherein said process essentially gives better purity & economy, handling and recyclability of resolving agent.

It has now been found that new conditions/process of the present invention makes it possible to convert Clopidogrel acid salt to free base in a single step in a non-aqueous condition, thereby, eliminating the occurrence of hydrolysis of ester leading to impurity of formula X. The process is simplifying the isolation of clopidogrel free base as well as resolving agent, making the process industrially more feasible.

Thus, according to the present invention, an improved synthesis of Clopidogrel is provided by reacting clopidogrel acid salt using ammonia in non-aqueous conditions to obtain clopidogrel in free base form. The reaction is performed in presence of an organic solvent. It should be understood that the present process is applicable to racemic clopidogrel as well as enantiomers of clopidogrel in any acid salt form.

The clopidogrel acid salts may be selected from known ones, for example bisulphate, hydrochloride, hydrobromide, naphthalene sulphonate, methane sulphonate, camphor sulphonate, tartarate etc. The ammonia may be employed in gaseous form or as a solution in suitable non-aqueous organic solvent. The organic solvent may be selected from any inert solvent, where a difference of solubility of ammonium salt and clopidogrel salt can be attained. Examples of solvents include, but not limited to, hydrocarbons, such as toluene, dichloromethane, dichloroethane, alcohols such as isopropanol, ethanol, ketones such as acetone, methyl ethylketone, Methylisobutyl ketone, methylpropylketone, etc., and ethers such as diisopropyl ether, diglyme, ter.butylmethyl ether, cyclopentylmethyl ether etc

In a preferred embodiment of the present invention the clopidogrel acid salt is reacted with ammonia gas in an organic solvent. The process is accomplished by making a solution or suspension of clopidogrel acid salt in an organic solution and passing ammonia gas to attain a constant pH in the range of about 8 to 9. Alternately an ammonia solution prepared in an organic solution (by passing ammonia into a neat solvent) may be added to a solution/suspension of clopidogrel salt in organic solvent or vise versa. The reaction may be conducted at room temperature, but preferably under cooling to control the exothermicity. While progressing the reaction the ammonium salt separates out from the organic solution and clopidogrel free base remain in solution in the organic solvent in stable form. The ammonium acid salt of camphor sulphonic acid may be conveniently removed/recovered from the reaction by simple filtration. The clopidogrel free base from the mother liquor is recovered after filtering out the ammonium salt of the camphor sulphonic acid from the organic solvent, followed by elimination of solvent. The yield of either clopidogrel free base or ammonium camphor sulphonate is nearly quantitative, whereas in conventional method it is about 75-90%.

The starting clopidogrel acid salt may be obtained by following any conventional methods. In the present invention, preferably the (S)-clopidogrel (L)-camphor sulphonate salt or bisulphate salt is used. (S)-clopidogrel (L)-camphor sulphonate salt is preferably obtained after resolution of racemic clopidogrel into it enantiomers using optically active camphorsulphonic acid. The process of resolution involves contacting Clopidogrel base with (−) camphor sulphonic acid in acetone or a mixture of polar and non-polar/weakly polar organic solvents and crystallizing the camphor sulphonic salt of (S)-clopidogrel according to our parent application publication number WO/2005/104663.

In another embodiment of the present invention the unwanted isomer, as (−) (R)-clopidogrel camphor sulphonate salt, left behind in the mother liquor is also treated with ammonia as described above to recover ammonium camphor sulphonate, and thereafter racemized and recycled by treatment with NaOH in alcoholic solvents like methanol at a temperature ranging from 30 to 50° C. to obtain a 50:50 ratio of both isomers (referred as racemic mixture) as exemplified in the parent application.

In yet another aspect, the present invention provides an efficient process to recover and recycle camphor sulphonic acid after its use in the resolution of a racemic substrate, for example, (±)-clopidogrel, comprises:

a) treating the diastereomeric camphor sulphonate salt of substrate with ammonia in a non-aqueous reaction solvent;
b) separating the ammonium camphor sulphonate salt from the substrate;
c) treating said ammonium camphor sulphonate salt with an acid in non-aqueous solvent to exchange the ammonium ion with the acid to free camphor sulphonic acid; and
d) isolating free camphor sulphonic acid from the reaction for recycling.

In the process according to the present invention, the recovery and recycling of camphorsulphonic acid comprises treating ammonium camphorsulphonate, obtained after following step (a) and (b) as illustrated for clopidogrel hereinbefore, in an organic solvent with an acid to liberate free camphor sulphonic acid and the by-product ammonium salt is removed by simple filtration. In the process ammonium camphor sulphonate salt was suspended or dissolved in an organic solvent for example, but limited to toluene, ethanol, isopropanol, acetone, methylethylketone, and treated with Hydrochloric acid, preferably in gaseous form or as a solution on organic solvent like Isopropanol until the salt exchange is completed. The free Camphor sulphonic acid can be isolated by filtering out the precipitated ammonium chloride and solvent elimination, followed by optional crystallization. The free Camphor sulphonic acid obtained after filtering out the precipitated ammonium chloride may be directly used as a solution, without further isolation, for resolution of clopidogrel, if the solvent is acetone or its mixtures with other solvents like dichloromethane.

Analytical characterization of the solid(s) obtained in accordance with the process of the invention was carried out by using X-ray powder diffraction using a PANALYTICAL XpertPRO X-Ray machine of Philips make. The X-ray powder diffraction patterns were recorded with Cu K alpha-1 radiation source (voltage of 50 kV; current: 25 mA).

The stable Clopidogrel bisulphate Form I obtained by the process of the present invention may be formulated into a dosage form, e.g., tablet, capsule, etc., by combining with one or more pharmaceutically acceptable excipients using known techniques. The resulting dosage form may include a suitable amount of the active ingredient required for the desired action. Further, the dosage form may be immediate release or extended release.

Further details of the process of the present invention will be apparent from the examples presented below. The examples presented are purely illustrative and are not limited to the particular embodiments illustrated herein but include the permutations, which are obvious as set forth in the description.

### EXAMPLES

### Example 1

### Form I Clopidogrel Hydrogen Sulphate

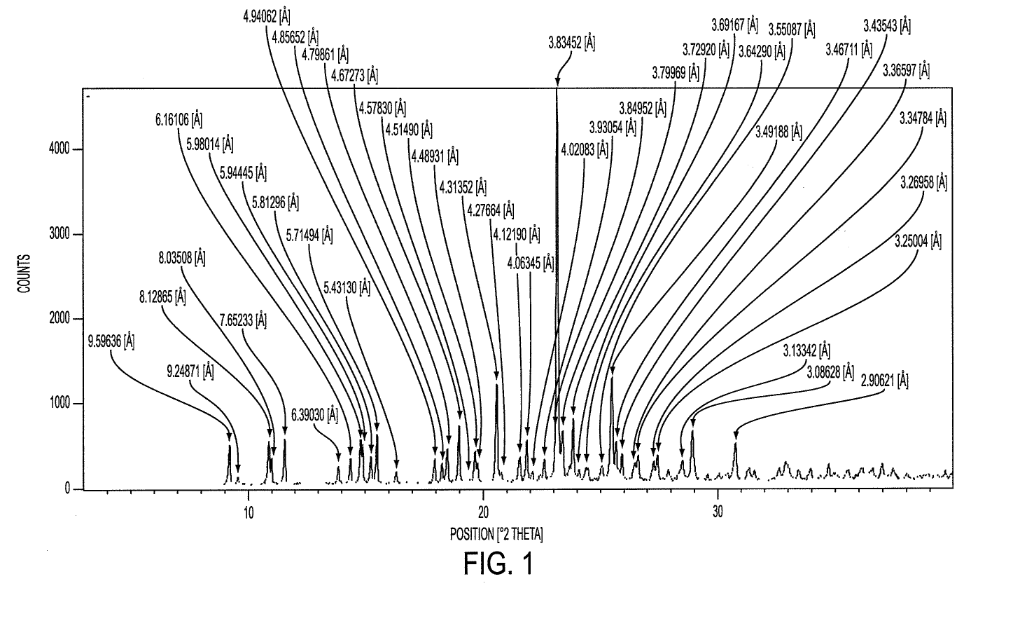

Clopidogrel base (100 gm) was dissolved in ethyl acetoacetate (600 ml) at room temperature. This mixture was cooled to −10° C. and concentrated sulphuric acid (98%, density=1.83) was added (15.5 gm) maintaining temperature −10° to 0° C. while addition. The reaction mass was stirred for 1.0 hour and warmed slowly to 10 to 15° C. in 30 to 45 minute. The formed crystals were stirred for 10 hours. The reaction mass temperature was further raised to 28 to 30° C. and maintained for 2 hours. The solid obtained was filtered under suction and washed with acetone, and dried in oven at 48° C. for 3 hours. The solid after drying weighed 96 gm, was Form I clopidogrel hydrogen sulphate (PXRD pattern incorporated: FIG. 1).

### Example 2

### Form I Clopidogrel Hydrogen Sulphate

Clopidogrel base (100 gm) was dissolved in 4-chloro-ethyl acetoacetate (600 ml) at room temperature. This mixture was cooled to −10° C. and concentrated sulphuric acid (98%, density=1.83) was added (15.5 gm) maintaining temperature −10° to 0° C. while addition. The reaction mass was stirred for 1.0 hour and warmed slowly to 10 to 15° C. in 30 to 45 minute. The formed crystals were stirred for 10 hours. The reaction mass temperature was further raised to 28 to 30° C. and maintained for 2 hours. The solid obtained was filtered under suction and washed with acetone, and dried in oven at 48° C. for 3 hours. The solid after drying weighed 95 gm, was Form I clopidogrel hydrogen sulphate (PXRD pattern incorporated: FIG. 1).

### Example 3

### Form I Clopidogrel Hydrogen Sulphate

Clopidogrel base (100 gm) was dissolved in a mixture of ethyl acetoacetate (500 ml) and acetone (100 ml) at room temperature. This mixture was cooled to −10° C. and concentrated sulphuric acid (98%, density=1.83) was added (15.5 gm) maintaining temperature at −10° to 0° C. while addition. The reaction mass was stirred for 1.0 hour and warmed slowly to 10 to 15° C. in 30 to 45 minute. The formed crystals were stirred for 10 hours. The reaction mass temperature was further raised to 28 to 30° C. and maintained for 2 hours. The solid obtained was filtered under suction and washed with acetone, and dried in oven at 48° C. for 3 hours. The solid after drying weighed 97 gm, was Form I clopidogrel hydrogen sulphate (PXRD pattern incorporated: FIG. 1).

### Example 4

### Form I Clopidogrel Hydrogen Sulphate

Clopidogrel base (5.79 kg) was dissolved in methyl ethyl ketone (37 liter) at room temperature. This mixture was cooled to −15° C. and concentrated sulphuric acid (96%, density=1.83) was added (1.02 liter) maintaining temperature −10° to 0° C. while addition. The reaction mass was stirred for 1.0 hour and warmed slowly to 10 to 15° C. for 30 to 45 minute. The formed crystals were stirred for 10 hours. The reaction mass temperature was further raised to 28 to 30° C. and maintained for 15 hours. The solid obtained was filtered under suction and washed with methyl ethyl ketone, and dried in oven at 48° C. for 3 hours. The solid after drying weighed 4.5 kg, was Form I clopidogrel hydrogen sulphate (PXRD pattern incorporated: FIG. 1).

### Example 5

### Form I Clopidogrel Hydrogen Sulphate

Clopidogrel base (5.79 kg) was dissolved in cyclopentylmethyl ether (37 liter) at room temperature. This mixture was cooled to −10° C. and concentrated sulphuric acid (96%, density=1.83) was added (1.02 liter) maintaining temperature −10° to 0° C. while addition. The reaction mass was stirred for 1.0 hour and warmed slowly to 10 to 15° C. in 30 to 45 minutes. The formed crystals were stirred for 7 hour. The reaction mass temperature was further raised to 28 to 30° C. and maintained for 8.0 hour. The solid obtained was filtered under suction and washed with cylcopentylmethylether, and dried in oven at 48° C. for 3 hour. The solid after drying weighed 6.82 kg (90%) was Form clopidogrel hydrogen sulphate (PXRD pattern is identical with FIG. 1).

### Example 6

Clopidogrel base (5.79 g) was dissolved in diisopropylglycol (60 ml) and acetone (10 ml) at room temperature. This mixture was cooled to −10° C. and concentrated sulphuric acid (96%, density=1.83) (1.02 ml) was added maintaining temperature −10° to −5° C. while addition. The reaction mass was stirred for 2.0 hour and warmed slowly to 10 to 15° C. in 30 to 45 minutes. The formed crystals were stirred for 7 hour. The reaction mass temperature was further raised to 28 to 30° C. and maintained for 8.0 hour. The solid obtained was filtered under suction and washed with cylcopentylmethylether, and dried in oven at 48° C. for 3 hour. The solid after drying weighed 5.2 gm was Form I clopidogrel hydrogen sulphate (PXRD pattern is identical with FIG. 1).

### Example 7

Clopidogrel base (5.79 g) was dissolved in methylpropylether (40 ml) and 10 ml methylpropylketone at room temperature. This mixture was cooled to −15° C. and concentrated sulphuric acid (1.02 ml) (96%, density=1.83) was added maintaining temperature −15° to −10° C. while addition. The reaction mass was stirred for 1.0 hour and warmed slowly to 10 to 15° C. in 30 to 45 minutes. The formed crystals were stirred for 4 hour. The reaction mass temperature was further raised to 28 to 30° C. and maintained for 10 hours. The solid obtained was filtered under suction and washed with methylpropylketone, and dried in oven at 48° C. for 3 hour. The solid after drying weighed 6.5 g was Form I clopidogrel hydrogen sulphate (PXRD pattern is identical with FIG. 1).

### Example 8

Clopidogrel base (5.79 g) was dissolved in t-butyl methyl ether (35 ml) and acetone (10 ml) at room temperature. This mixture was cooled to −15° C. and concentrated sulphuric acid (1.02 L) (96%, density=1.83) was added maintaining temperature −10° to 0° C. while addition. The reaction mass was stirred for 1.0 hour and warmed slowly to 10 to 15° C. in 1 hour. The formed crystals were stirred for 5 hours. The reaction mass temperature was further raised to 28 to 30° C. and maintained for 3.0 hours. The solid obtained was filtered under suction and washed with t-butyl methyl ether, and dried in oven at 48° C. for 3 hour. The solid after drying weighed 6.7 kg (90%) was Form I clopidogrel hydrogen sulphate (PXRD pattern is identical with FIG. 1).

### Example 9

Clopidogrel base (5.5 g) was dissolved in propylmethyl cellosolve (40 ml) at room temperature. This mixture was cooled to −10° C. and concentrated sulphuric acid (1.1 ml) (90%) was added maintaining temperature −10° to 0° C. while addition. The reaction mass was stirred for 1.0 hour and warmed slowly to 15 to 20° C. in 2 hours. The formed crystals were stirred for 3 hour. The reaction mass temperature was further raised to 28 to 30° C. and maintained for 8.0 hour. The solid obtained was filtered under suction and washed with acetone, and dried in oven at 48° C. for 3 hour. The solid after drying weighed 5.9 g was Form I clopidogrel hydrogen sulphate (PXRD pattern is identical with FIG. 1).

### Example 10

### (S)(+)Clopidogrel Base

93.0 gm (0.28 mole) of racemic base methyl-2-(2-chlorophenyl)-2-(4,5,6,7-tetrahydrothieno[3,2-c]pyridin-5-yl) acetate was charged in 550 ml of mixture of acetone and dichloromethane. 73.8 gm (0.31 mole) levo-camphor-10-sulphonic acid was added in the solution. The clear solution was stirred overnight at 30±2° C. and cooled the reaction mass to −2 to 3° C. The crystals obtained was filtered and washed with acetone and dried at room temperature under vacuum to give 61 gm of diastereomeric (S)-clopidogrel camphor sulphonate salt.

(S)-clopidogrel camphor sulphonate salt (60 gm) obtained above was dissolved suspended in dichloromethane at room temperature. The solution suspension is cooled to about 15-20 degrees and passed ammonia gas until a constant pH of 8.5 to 9.0 is reached. The solution was stirred for 3 hours and filtered to obtain ammonium camphor sulphonate which is collected for recycling. The clopidogrel free base is recovered from the mother liquor by concentrating under vacuum to obtain clopidogrel free base in syrup form.

### Example 11

### (S)(+)Clopidogrel Base

150.0 gm (0.28 mole) of racemic methyl-2-(2-chlorophenyl)-2-(4,5,6,7-tetrahydrothieno[3,2-c]pyridin-5-yl) acetate bisulphate was charged in 600 ml dichloromethane at about 20 degrees. To this mixture ammonia gas is passed until a constant pH of 8.5 to 9 is reached. The suspension was stirred for 3 hours, filtered to remove insoluble salt. The dichloromethane solution was concentrated to 200 ml and added 350 ml acetone. 73.8 gm (0.31 mole) levo-camphor-10-sulphonic acid was added to the solution. The clear solution was stirred for 10 hours at 30±2° C. The reaction mass was cooled to −2 to 3° C. The crystals obtained was filtered, washed with acetone and dried under reduced pressure to give (S)(+)-clopidogrel camphor sulphonate. The yield obtained is 80% on the basis of the starting racemate charged. The crystals have [α]_{D}20+24.49 (c=1.89%, methanol); HPLC (AGP® column) assay=99.285%.

The (S)(+)-clopidogrel camphor sulphonate was further taken in dichloromethane (500 ml). To this suspension a solution of 50 ml isopropanol-ammonia solution was added slowly while keeping the reaction mass at about 15-20 degrees C. The mixture was stirred for 2 hours and the precipitated ammonium camphor sulphonate was isolated by filtration. The dichloromethane solution was washed with water, dried over sodium sulphate and concentrated to dryness under vacuum to obtain (S)(+)clopidogrel base.

### Example 12

Clopidogrel base (5.79 kg) was dissolved in methyl isopropyl ketone (37 liter) at room temperature. This mixture was cooled to −10° C. and concentrated sulphuric acid (96%, density=1.83) was added (1.02 liter) maintaining temperature −10° to 0° C. while addition. The reaction mass was stirred for 1.0 hour and warmed slowly to 10 to 15° C. in 30 to 45 minutes. The formed crystals were stirred for 7 hour. The reaction mass temperature was further raised to 28 to 30° C. and maintained for 8.0 hour. The solid obtained was filtered under suction and washed with methyl isopropyl ketone, and dried in oven at 48° C. for 3 hour. The solid after drying weighed 6.82 kg (90%) was Form I clopidogrel hydrogen sulphate (PXRD pattern is identical with FIG. 1).

### Example 13

### Recovery of Camphor Sulphonic Acid from Mother Liquor after Resolution

The mother liquor comprising mixture of (R)-Clopidogrel (enriched) & (S)-clopidogrel camphor sulphonate salt obtained in Example 10 or 11 after separation of pure (S)-clopidogrel camphor sulphonate salt was evaporated to dryness. To this 500 ml dichloromethane was added and passed gaseous ammonia while maintaining temperature about 15-20 degrees C. to reach a pH of 9. The suspension was stirred for 2 hours, filtered and washed with dichloromethane to obtain ammonium camphorsulphonate.

### Example 14

### Recovery of Camphorsulphonic Acid

175.0 gm of ammonium camphor sulphonate was charged in 1200 ml isopropanol HCl solution (concentration of HCl was about 18-20%) at about 20 degrees. The mixture is stirred for 3 hours at room temperature and the precipitated ammonium chloride was filtered out. The filtrate was distilled out under vacuum and 200 ml Isopropanol and 240 ml toluene were added. The mixture was stirred for additional 1 hour and filtered to obtain 148 gm fresh camphor sulphonic acid.

### Example 15

### Recovery & Recycling of Camphorsulphonic Acid In Situ

175.0 gm of ammonium camphor sulphonate was charged in 875 ml acetone and purged Hydrochloric acid gas till a pH of 1-2 was obtained at about 20 degrees. The mixture was stirred for 3 hours at room temperature and the precipitated ammonium chloride was filtered out. The filtrate was distilled out under vacuum and 900 ml acetone and 182 gm racemic clopidogrel were added. The mixture was stirred for additional 12 hour at room temperature and further cooled to about 0.-5 degree Celsius. The mixture was maintained for further 5 hours at 0-5 degree Celsius, filtered, washed with acetone and dried under vacuum to obtain 110 gm (S)(+)-clopidogrel L-camphor sulphonate salt.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1-23. (canceled)

24. A process for recovery and recycling of camphorsulphonic acid after its use in the resolution of a racemic substrate, the process comprising the steps of:
a) treating diastereomeric camphor sulphonate salt of the substrate with ammonia in a non-aqueous solvent to form ammonium camphor sulphonate salt;
b) separating the ammonium camphor sulphonate salt from the substrate;
c) treating said ammonium camphor sulphonate salt with an acid in a non-aqueous solvent to exchange the ammonium ion with the acid to produce free camphor sulphonic acid; and
d) isolating free camphor sulphonic acid from the reaction for recycling.

25. The process as claimed in claim 24, wherein the substrate is clopidogrel.

26. The process as claimed in claim 24, wherein diastereomeric salt of the substrate is clopidogrel L-camphor sulphonic acid salt.

27. The process as claimed in claim 24, wherein the non-aqueous solvent is acetone, isopropanol, toluene, or mixtures thereof.
